SEQUENCE LISTING (12 sequences)

SEQ ID NO: 1 - protein, 1967 aa, Artificial Sequence
  PLASMID pTrcCFRET3
ASDMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAALQSSGGGGGSTMGGSHHHHHHGMASMTGGQQMGRDLYDDDDKHRWIRPRGSSGGGGSGGGGSGGGSSRMVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGKLGCFGGXEKIFSLIQIKSERRSGLIKQNLPGGSSAVVPPDPMPNSEVKRRSADGSVGSPHARVGNCQASNKTKGSVERLGLSFYLLFVGERSPEXDKSAGSGFERCEATARRVAGRTPAINCQASNXAEGHPDGWPFCVSTNSFCLFFXIHSNMYPLMRQXPXXMLQXYXKRKSMSIQHFRVALIPFFAAFCLPVFAHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWXLSDQVYSYILXIDLKLHFXFKRIXVKILFDNLMTKIPXREFSFHXASDPVEKIKGSSXDPFFLRVICCLQTKKPPLPAVVCLPDQELPTLFPKVTGFSRAQIPNTVLLVXPXLGHHFKNSVAPPTYLALLILLPVAAASGDKSCLTGLDSRRXLPDKAQRSGXTGGSCTQPSLERTTYTELRYLQRELXESATLPEGRKADRYPVSGRVGTGERTRELPGGNAWYLYSPVGFRHLXLERRFLXCSSGGRSLWKNASNAAFLRFLAFCWPFAHMFFPALSPDSVDNRITAFEXADTARRSRTTERSESVSEEAEERLMRYFLLTHLCGISHRIWCTLSTICSDAAXLSQYTLRYRYVTGSWLRPDTRQHPLTRPDGLVCSRHPLTDKLXPSPGAACVRGFHRHHRNARGSRSIRARRRSGMHLRXHHRMVQNLSRYGMIAPGRESIQGGECETSNVIRCRRVCRCLLSDRFPRGEPGQPRFCENAGKSGSGDGGAELHSQPRGTTTGGQTVVADWRCHLQSGPARAVANCRGDXISRRSTGCQRGGVDGRTKRRRSLXSGGAQSSRATRQWADHXLSAGXPGCHCCGSCLHXCSGVISXCLXPDTHQQYYFLPXRRYATGRGASGRIGSPANRAVSGPIKFCLGASASGWLAXISHSQSNSADSGTGRRLECHVRFSTNHANAEXGHRSHCDAGCQRSDGAGRNARHYRVRAARWCGYLGSGIRRYRRQLMLYPAVNHHQTGFSPAGANQRGPLAATLSGPGGEGQSAVARLTGEKKNHPGAQYANRLSPRVGRFINAAGTTGFPTGKRAVSATQLMXVSANXSGLTAYHRLHGAPMLLASGSHRKLWYGCAGRKSLHNSCRSRRTPVLDNVFCADIITVLANILKXAVDNXSSGSYNVWNCERITISHRKQRRXEKAKRHCSLTIYQTICVGTRPELSINFIIKNXRGIYXCIDXIRRNKPCRDLQLVPYGNS

SEQ ID NO: 2 - DNA, 11806 bp, Artificial Sequence
  PLASMID pTrcCFRET3
agcttccgacatggtgagcaagggcgaggagctgttcaccggggtggtgcccatcctggttcgaaggctgtaccactcgttcccgctcctcgacaagtggccccaccacgggtaggaccacgagctggacggcgacgtaaacggccacaagttcagcgtgtccggcgagggcgagggcgagctcgacctgccgctgcatttgccggtgttcaagtcgcacaggccgctcccgctcccgcttgccacctacggcaagctgaccctgaagttcatctgcaccaccggcaagctgcccgtgccacggtggatgccgttcgactgggacttcaagtagacgtggtggccgttcgacgggcacggctggcccaccctcgtgaccaccctgacctggggcgtgcagtgcttcagccgctaccccgagaccgggtgggagcactggtgggactggaccccgcacgtcacgaagtcggcgatggggctccacatgaagcagcacgacttcttcaagtccgccatgcccgaaggctacgtccaggagcgggtgtacttcgtcgtgctgaagaagttcaggcggtacgggcttccgatgcaggtcctcgccaccatcttcttcaaggacgacggcaactacaagacccgcgccgaggtgaagttcgaggggtggtagaagaagttcctgctgccgttgatgttctgggcgcggctccacttcaagctccccgacaccctggtgaaccgcatcgagctgaagggcatcgacttcaaggaggacggcaacatgctgtgggaccacttggcgtagctcgacttcccgtagctgaagttcctcctgccgttgtacctggggcacaagctggagtacaactacatcagccacaacgtctatatcaccgccgacaaggaccccgtgttcgacctcatgttgatgtagtcggtgttgcagatatagtggcggctgttgcagaagaacggcatcaaggccaacttcaagatccgccacaacatcgaggacggcagcgtcgtcttcttgccgtagttccggttgaagttctaggcggtgttgtagctcctgccgtcgcagcagctcgccgaccactaccagcagaacacccccatcggcgacggccccgtgctgctgcccgtcgagcggctggtgatggtcgtcttgtgggggtagccgctgccggggcacgacgacggcgacaaccactacctgagcacccagtccgccctgagcaaagaccccaacgagaagcgcgagctgttggtgatggactcgtgggtcaggcgggactcgtttctggggttgctcttcgcgcttcacatggtcctgctggagttcgtgaccgccgccctgcagtcctcagggggagggggcggagtgtaccaggacgacctcaagcactggcggcgggacgtcaggagtccccctcccccgccttccaccatggggggttctcatcatcatcatcatcatggtatggctagcatgactggtggaaggtggtaccccccaagagtagtagtagtagtagtaccataccgatcgtactgaccaccacagcaaatgggtcgggatctgtacgacgatgacgataagcatcgatggatccgacctcgtgtcgtttacccagccctagacatgctgctactgctattcgtagctacctaggctggagcaggttcctcagggggagggggatctggaggcggaggctctggcggtggctcctctagaattccaaggagtccccctccccctagacctccgcctccgagaccgccaccgaggagatcttaggtgagcaagggcgaggagctgttcaccggggtggtgcccatcctggtcgagctggacggccactcgttcccgctcctcgacaagtggccccaccacgggtaggaccagctcgacctgcccgacgtaaacggccacaagttcagcgtgtccggcgagggcgagggcgatgccacctacgggctgcatttgccggtgttcaagtcgcacaggccgctcccgctcccgctacggtggatgcccaagctgaccctgaagttcatctgcaccaccggcaagctgcccgtgccctggcccaccctgttcgactgggacttcaagtagacgtggtggccgttcgacgggcacgggaccgggtgggacgtgaccaccttcggctacggcctgcagtgcttcgcccgctaccccgaccacatgaagcagcactggtggaagccgatgccggacgtcacgaagcgggcgatggggctggtgtacttcgtgcacgacttcttcaagtccgccatgcccgaaggctacgtccaggagcgcaccatcttcttcgtgctgaagaagttcaggcggtacgggcttccgatgcaggtcctcgcgtggtagaagaacaaggacgacggcaactacaagacccgcgccgaggtgaagttcgagggcgacaccctggtgttcctgctgccgttgatgttctgggcgcggctccacttcaagctcccgctgtgggaccagaaccgcatcgagctgaagggcatcgacttcaaggaggacggcaacatcctggggcacaacttggcgtagctcgacttcccgtagctgaagttcctcctgccgttgtaggaccccgtgttgctggagtacaactacaacagccacaacgtctatatcatggccgacaagcagaagaacggcgacctcatgttgatgttgtcggtgttgcagatatagtaccggctgttcgtcttcttgcccatcaaggtgaacttcaagatccgccacaacatcgaggacggcagcgtgcagctcgccgagtagttccacttgaagttctaggcggtgttgtagctcctgccgtcgcacgtcgagcggctccactaccagcagaacacccccatcggcgacggccccgtgctgctgcccgacaaccactaggtgatggtcgtcttgtgggggtagccgctgccggggcacgacgacgggctgttggtgatcctgagctaccagtccgccctgagcaaagaccccaacgagaagcgcgatcacatggtcctggactcgatggtcaggcgggactcgtttctggggttgctcttcgcgctagtgtaccaggagctggagttcgtgaccgccgccgggatcactctcggcaagcttggctgttttggcggatgcgacctcaagcactggcggcggccctagtgagagccgttcgaaccgacaaaaccgcctacagagaagattttcagcctgatacagattaaatcagaacgcagaagcggtctgataaaacatctcttctaaaagtcggactatgtctaatttagtcttgcgtcttcgccagactattttgtgaatttgcctggcggcagtagcgcggtggtcccacctgaccccatgccgaactcagaagtcttaaacggaccgccgtcatcgcgccaccagggtggactggggtacggcttgagtcttcagaaacgccgtagcgccgatggtagtgtggggtctccccatgcgagagtagggaactgccactttgcggcatcgcggctaccatcacaccccagaggggtacgctctcatcccttgacggtggcatcaaataaaacgaaaggctcagtcgaaagactgggcctttcgttttatctgttgttccgtagtttattttgctttccgagtcagctttctgacccggaaagcaaaatagacaacaatgtcggtgaacgctctcctgagtaggacaaatccgccgggagcggatttgaacgttgcgaacagccacttgcgagaggactcatcctgtttaggcggccctcgcctaaacttgcaacgctagcaacggcccggagggtggcgggcaggacgcccgccataaactgccaggcatcaaattatcgttgccgggcctcccaccgcccgtcctgcgggcggtatttgacggtccgtagtttaatagcagaaggccatcctgacggatggcctttttgcgtttctacaaactctttttgtttatttcgtcttccggtaggactgcctaccggaaaaacgcaaagatgtttgagaaaaacaaataatttctaaatacattcaaatatgtatccgctcatgagacaataaccctgataaatgcttcaaaagatttatgtaagtttatacataggcgagtactctgttattgggactatttacgaagtataatattgaaaaaggaagagtatgagtattcaacatttccgtgtcgcccttattccctttattataactttttccttctcatactcataagttgtaaaggcacagcgggaataagggaattttgcggcattttgccttcctgtttttgctcacccagaaacgctggtgaaagtaaaagaaaaacgccgtaaaacggaaggacaaaaacgagtgggtctttgcgaccactttcattttcttgctgaagatcagttgggtgcacgagtgggttacatcgaactggatctcaacagcggtaaacgacttctagtcaacccacgtgctcacccaatgtagcttgacctagagttgtcgccattgatccttgagagttttcgccccgaagaacgttttccaatgatgagcacttttaaagttctctaggaactctcaaaagcggggcttcttgcaaaaggttactactcgtgaaaatttcaagagctatgtggcgcggtattatcccgtgttgacgccgggcaagagcaactcggtcgccgcatcgatacaccgcgccataatagggcacaactgcggcccgttctcgttgagccagcggcgtaacactattctcagaatgacttggttgagtactcaccagtcacagaaaagcatcttacggatgtgataagagtcttactgaaccaactcatgagtggtcagtgtcttttcgtagaatgccttggcatgacagtaagagaattatgcagtgctgccataaccatgagtgataacactgcggcaccgtactgtcattctcttaatacgtcacgacggtattggtactcactattgtgacgccgcaacttacttctgacaacgatcggaggaccgaaggagctaaccgcttttttgcacaacatgttgaatgaagactgttgctagcctcctggcttcctcgattggcgaaaaaacgtgttgtagggggatcatgtaactcgccttgatcgttgggaaccggagctgaatgaagccataccaaaccccctagtacattgagcggaactagcaacccttggcctcgacttacttcggtatggtttcgacgagcgtgacaccacgatgcctgtagcaatggcaacaacgttgcgcaaactattaacgctgctcgcactgtggtgctacggacatcgttaccgttgttgcaacgcgtttgataattgtggcgaactacttactctagcttcccggcaacaattaatagactggatggaggcggataaaccgcttgatgaatgagatcgaagggccgttgttaattatctgacctacctccgcctattagttgcaggaccacttctgcgctcggcccttccggctggctggtttattgctgataaatctcaacgtcctggtgaagacgcgagccgggaaggccgaccgaccaaataacgactatttagtggagccggtgagcgtgggtctcgcggtatcattgcagcactggggccagatggtaagccacctcggccactcgcacccagagcgccatagtaacgtcgtgaccccggtctaccattcggctcccgtatcgtagttatctacacgacggggagtcaggcaactatggatgaacgaaataggagggcatagcatcaatagatgtgctgcccctcagtccgttgatacctacttgctttatcacagatcgctgagataggtgcctcactgattaagcattggtaactgtcagaccaagtttatgtctagcgactctatccacggagtgactaattcgtaaccattgacagtctggttcaaatctcatatatactttagattgatttaaaacttcatttttaatttaaaaggatctaggtgaagagtatatatgaaatctaactaaattttgaagtaaaaattaaattttcctagatccacttgatcctttttgataatctcatgaccaaaatcccttaacgtgagttttcgttccactgagcctaggaaaaactattagagtactggttttagggaattgcactcaaaagcaaggtgactcggtcagaccccgtagaaaagatcaaaggatcttcttgagatcctttttttctgcgcgtaatcagtctggggcatcttttctagtttcctagaagaactctaggaaaaaaagacgcgcattactgctgcttgcaaacaaaaaaaccaccgctaccagcggtggtttgtttgccggatcaagagacgacgaacgtttgtttttttggtggcgatggtcgccaccaaacaaacggcctagttctgctaccaactctttttccgaaggtaactggcttcagcagagcgcagataccaaatactgtcgatggttgagaaaaaggcttccattgaccgaagtcgtctcgcgtctatggtttatgacaccttctagtgtagccgtagttaggccaccacttcaagaactctgtagcaccgcctacataggaagatcacatcggcatcaatccggtggtgaagttcttgagacatcgtggcggatgtatcctcgctctgctaatcctgttaccagtggctgctgccagtggcgataagtcgtgtcttacggagcgagacgattaggacaatggtcaccgacgacggtcaccgctattcagcacagaatgcgggttggactcaagacgatagttaccggataaggcgcagcggtcgggctgaacgggggggcccaacctgagttctgctatcaatggcctattccgcgtcgccagcccgacttgccccccttcgtgcacacagcccagcttggagcgaacgacctacaccgaactgagatacctacagcgaagcacgtgtgtcgggtcgaacctcgcttgctggatgtggcttgactctatggatgtcgctgagctatgagaaagcgccacgcttcccgaagggagaaaggcggacaggtatccggtaagactcgatactctttcgcggtgcgaagggcttccctctttccgcctgtccataggccattccggcagggtcggaacaggagagcgcacgagggagcttccagggggaaacgcctggtatctgccgtcccagccttgtcctctcgcgtgctccctcgaaggtccccctttgcggaccatagattatagtcctgtcgggtttcgccacctctgacttgagcgtcgatttttgtgatgctcgtcaatatcaggacagcccaaagcggtggagactgaactcgcagctaaaaacactacgagcagaggggggcggagcctatggaaaaacgccagcaacgcggcctttttacggttcctggcctttccccccgcctcggatacctttttgcggtcgttgcgccggaaaaatgccaaggaccggaattgctggccttttgctcacatgttctttcctgcgttatcccctgattctgtggataaccgaacgaccggaaaacgagtgtacaagaaaggacgcaataggggactaagacacctattggctattaccgcctttgagtgagctgataccgctcgccgcagccgaacgaccgagcgcagcgaataatggcggaaactcactcgactatggcgagcggcgtcggcttgctggctcgcgtcgctgtcagtgagcgaggaagcggaagagcgcctgatgcggtattttctccttacgcatctgtgcagtcactcgctccttcgccttctcgcggactacgccataaaagaggaatgcgtagacaccggtatttcacaccgcatatggtgcactctcagtacaatctgctctgatgccgcatagttgccataaagtgtggcgtataccacgtgagagtcatgttagacgagactacggcgtatcaaaagccagtatacactccgctatcgctacgtgactgggtcatggctgcgccccgacacccgttcggtcatatgtgaggcgatagcgatgcactgacccagtaccgacgcggggctgtgggcccaacacccgctgacgcgccctgacgggcttgtctgctcccggcatccgcttacagacaaggttgtgggcgactgcgcgggactgcccgaacagacgagggccgtaggcgaatgtctgttgctgtgaccgtctccgggagctgcatgtgtcagaggttttcaccgtcatcaccgaaacgccgacactggcagaggccctcgacgtacacagtctccaaaagtggcagtagtggctttgcggcgaggcagcagatcaattcgcgcgcgaaggcgaagcggcatgcatttacgttgacaccacgctccgtcgtctagttaagcgcgcgcttccgcttcgccgtacgtaaatgcaactgtggttcgaatggtgcaaaacctttcgcggtatggcatgatagcgcccggaagagagtcaattcaagcttaccacgttttggaaagcgccataccgtactatcgcgggccttctctcagttaagtgggtggtgaatgtgaaaccagtaacgttatacgatgtcgcagagtatgccggtgtctcttcccaccacttacactttggtcattgcaatatgctacagcgtctcatacggccacagagaaatcagaccgtttcccgcgtggtgaaccaggccagccacgtttctgcgaaaacgcgggaaatagtctggcaaagggcgcaccacttggtccggtcggtgcaaagacgcttttgcgccctttaagtggaagcggcgatggcggagctgaattacattcccaaccgcgtggcacaacaactggttcaccttcgccgctaccgcctcgacttaatgtaagggttggcgcaccgtgttgttgacccgggcaaacagtcgttgctgattggcgttgccacctccagtctggccctgcacgcgccgtgcccgtttgtcagcaacgactaaccgcaacggtggaggtcagaccgggacgtgcgcggcacgcaaattgtcgcggcgattaaatctcgcgccgatcaactgggtgccagcgtggtggtgtgcgtttaacagcgccgctaatttagagcgcggctagttgacccacggtcgcaccaccacacgatggtagaacgaagcggcgtcgaagcctgtaaagcggcggtgcacaatcttctcgcgcgctaccatcttgcttcgccgcagcttcggacatttcgccgccacgtgttagaagagcgcgaacgcgtcagtgggctgatcattaactatccgctggatgaccaggatgccattgctgtggttgcgcagtcacccgactagtaattgataggcgacctactggtcctacggtaacgacaccaagctgcctgcactaatgttccggcgttatttcttgatgtctctgaccagacacccatcattcgacggacgtgattacaaggccgcaataaagaactacagagactggtctgtgggtagtacagtattattttctcccatgaagacggtacgcgactgggcgtggagcatctggtcgcattgtcataataaaagagggtacttctgccatgcgctgacccgcacctcgtagaccagcgtatgggtcaccagcaaatcgcgctgttagcgggcccattaagttctgtctcggcgcgtctgcacccagtggtcgtttagcgcgacaatcgcccgggtaattcaagacagagccgcgcagacggtctggctggctggcataaatatctcactcgcaatcaaattcagccgatagcggaacgggcagaccgaccgaccgtatttatagagtgagcgttagtttaagtcggctatcgccttgcccaaggcgactggagtgccatgtccggttttcaacaaaccatgcaaatgctgaatgagggcattccgctgacctcacggtacaggccaaaagttgtttggtacgtttacgacttactcccgttcgttcccactgcgatgctggttgccaacgatcagatggcgctgggcgcaatgcgcgccaagcaagggtgacgctacgaccaacggttgctagtctaccgcgacccgcgttacgcgcggtttaccgagtccgggctgcgcgttggtgcggatatctcggtagtgggatacgacgataccgaatggctcaggcccgacgcgcaaccacgcctatagagccatcaccctatgctgctatggcaagacagctcatgttatatcccgccgtcaaccaccatcaaacaggattttcgcctgctggttctgtcgagtacaatatagggcggcagttggtggtagtttgtcctaaaagcggacgaccggcaaaccagcgtggaccgcttgctgcaactctctcagggccaggcggtgaagggcaatcccgtttggtcgcacctggcgaacgacgttgagagagtcccggtccgccacttcccgttagagctgttgcccgtctcactggtgaaaagaaaaaccaccctggcgcccaatacgcaaaccgtcgacaacgggcagagtgaccacttttctttttggtgggaccgcgggttatgcgtttggccctctccccgcgcgttggccgattcattaatgcagctggcacgacaggtttcccgactggggagaggggcgcgcaaccggctaagtaattacgtcgaccgtgctgtccaaagggctgaccaaagcgggcagtgagcgcaacgcaattaatgtgagttagcgcgaattgatctggtttgactttcgcccgtcactcgcgttgcgttaattacactcaatcgcgcttaactagaccaaactgagcttatcatcgactgcacggtgcaccaatgcttctggcgtcaggcagccatcggaagcttcgaatagtagctgacgtgccacgtggttacgaagaccgcagtccgtcggtagccttcgagtggtatggctgtgcaggtcgtaaatcactgcataattcgtgtcgctcaaggcgcactcccaccataccgacacgtccagcatttagtgacgtattaagcacagcgagttccgcgtgaggcgttctggataatgttttttgcgccgacatcataacggttctggcaaatattctgaaatggcaagacctattacaaaaaacgcggctgtagtattgccaagaccgtttataagactttacagctgttgacaattaatcatccggctcgtataatgtgtggaattgtgagcggataacaattcgacaactgttaattagtaggccgagcatattacacaccttaacactcgcctattgttattcacacaggaaacagcgccgctgagaaaaagcgaagcggcactgctctttaacaatttaaagtgtgtcctttgtcgcggcgactctttttcgcttcgccgtgacgagaaattgttaaattcagacaatctgtgtgggcactcgaccggaattatcgattaactttattattaaaaattaagtctgttagacacacccgtgagctggccttaatagctaattgaaataataatttttaataagaggtatatattaatgtatcgattaaataaggaggaataaaccatgtcgagatctgcattctccatatataattacatagctaatttattcctccttatttggtacagctctagacgtgctggtaccatatgggaattcgacgaccatggtatacccttaagct

SEQ ID NO: 3 - protein, 4 aa, Artificial Sequence
  Linker Sequence
SGGG

SEQ ID NO: 4 - protein, 8 aa, Artificial Sequence
  Linker Sequence
SGGGSGGG

SEQ ID NO: 5 - protein, 12 aa, Artificial Sequence
  Linker Sequence
SGGGSGGGSGGG

SEQ ID NO: 6 - protein, 16 aa, Artificial Sequence
  Linker Sequence
SGGGSGGGSGGGSGGG

SEQ ID NO: 7 - protein, 15 aa, Probe
SSNHQSSRLIELLSR

SEQ ID NO: 8 - protein, 14 aa, Probe
SAPRATISHYLMGG

SEQ ID NO: 9 - protein, 15 aa, Probe
SSPGSREWFKDMLSR

SEQ ID NO: 10 - DNA, 10 bp, Artificial Sequence
  Translation Initiation Site
gccaccatgg

SEQ ID NO: 11 - DNA, 10 bp, Artificial Sequence
  Translation Initiation Site
gcccccatgg

SEQ ID NO: 12 - protein, 17 aa, Artificial Sequence
  Biotin Acceptance Sequence
MSGLNDIFEAQKIEWHE

(19) United States
(12) Patent Application Publication
(10) Pub. No.: US 2004/0265902 A1
(43) Pub. Date: Dec. 30, 2004

(54) Universatl fluorescent sensors

(76) Inventors: Mark David Fricker, Oxford (GB); David John Talbut Vaux, Oxford (GB)

Correspondence Address: FULBRIGHT & JAWORSKI, LLP, 1301 MCKINNEY, SUITE 5100, HOUSTON, TX, 77010-3095, US

(21) Appl. No.: 10/477,044

(22) PCT Filed: May 10, 2002
(86) PCT No.: PCT/GB02/02183

(30) Foreign Application Priority Data
May 10, 2001 (GB) 0111459.4

(51) Int. Cl. G01N 33/53; C12Q 1/37; C07K 16/46; C07K 14/47

(52) U.S. Cl. 435/7.1; 435/23; 530/409; 530/391.1

(57) ABSTRACT

A probe comprises: (1) a target binding site moiety which is attached to a first fluorescent polypeptide; (ii) a mimic moiety which is capable of binding to the target binding site moiety and is attached to a second fluorescent polypeptide; and (iii) a linker which connects the two fluorescent polypeptides and which allows the distance between said fluorescent polypeptides to vary, said fluorescent polypeptides being so as to display fluorescence resonance energy transfer (FRET) between them, wherein the linker comprises one or more of: (1) a sequence capable of being recognised and bound by an immobilized component; (2) a protease cleavage site; (3) a non-analyte binding site; (4) two or more copies of the sequence (SerGly₃); or (5) one or more copies of a rod domain from a structural protein. Probes of the invention are used, for example, in the detection of a wide range of substances or in the identification of inhibitors of the interaction between two substances which, in the absence of an inhibitor, interact with each other.

### FIELD OF THE INVENTION

The invention relates to probes which are used for the detection of a wide range of substances. The invention also relates to probes which are used for the identification of inhibitors which reduce binding between two substances, which two substances bind to each other in the absence of an inhibitor.

Probes of the invention can be used in, for example, medical diagnosis, the detection of pollutants in water systems and the detection of contaminants in foodstuffs and in animal and plant biology. They can also be used in the identification of new therapeutic substances.

### BACKGROUND TO THE INVENTION

When a fluorescent molecule absorbs light, an electron is excited to a higher energy level. Typically the electron loses some energy before decaying back to ground state. During this transition, a photon is emitted with less energy than the excitation photon and hence at a longer wavelength. If a second fluorophor is in close proximity, the energy released by the electron as it decays in the donor fluorophor may be transferred directly to the second acceptor fluorophor and excite one of the electrons of the latter to a higher energy level. When the electron in the acceptor decays from this state, an even longer wavelength photon is released. The process is termed fluorescence resonance energy transfer (FRET). The extent to which FRET takes place is critically dependent on the overlap of the spectra between the two fluorophors and their separation. Thus, FRET decreases roughly in proportion to the sixth power of the separation between the two fluorophors and is a powerful reporter for the separation of the two fluorophors at the molecular level.

The coding sequences for a range of fluorescent proteins are now available and some of these proteins have an appropriate overlap in their emission and excitation spectra for efficient FRET to take place. Heim and Tsien (1996, Curr. Biol. 6, 178-182) have demonstrated that FRET can occur between two such fluorescent proteins when they are tethered together and that the FRET signal alters if the peptide linker is severed by a protease. Using this principle Miyawaki et al. (1997, Nature 388, 882-887) demonstrated the use of a FRET-based indicator for calcium detection. This was achieved by using the calcium-binding protein (calmodulin) and a short calmodulin-binding target sequence (M13) as part of the linker between the two fluorophors. Calmodulin undergoes a conformational change on binding calcium and subsequently binds to the adjacent calmodulin-binding sequence. This serves to alter the separation of the fluorescent proteins and modulates the level of FRET.

Although the potential exists to generate probes for other molecules, identification and screening of proteins or protein motifs with appropriate properties to both bind to the target and to alter the separation of the fluorophors is not straightforward.

### SUMMARY OF THE INVENTION

According to the invention there is provided a probe comprising:

(i) a target binding site moiety which is attached to a first fluorescent polypeptide;

(ii) a mimic moiety which is capable of binding to the target binding site moiety and is attached to a second fluorescent polypeptide; and

(iii) a linker which connects the two fluorescent polypeptides and which allows the distance between said fluorescent polypeptides to vary, said fluorescent polypeptides being so as to display fluorescence resonance energy transfer (FRET) between them, wherein the linker comprises one or more of: (1) a sequence capable of being recognised and bound by an immobilized component; (2) a protease cleavage site; (3) a non-analyte binding site; (4) two or more copies of the sequence (SerGly₃); or (5) one or more copies of a rod domain from a structural protein.

The invention also provides:

a polynucleotide which encodes a probe of the invention;

a vector incorporating a polynucleotide of the invention;

a cell harbouring a probe, polynucleotide or vector of the invention;

a fungus, plant or animal comprising a probe, polynucleotide, vector or cell of the invention;

a sensor comprising:

(i) a probe of the invention;

(ii) a light source which is capable of exciting the probe; and

(iii) a detector which is capable of measuring the amount of FRET from the probe;

a method for detecting the presence or absence of a target substance in a test sample comprising:

(i) providing a probe, cell or sensor of the invention, wherein the target binding site moiety of the probe, cell or sensor is capable of binding to the target substance;

(ii) determining the amount of FRET of the probe, cell or sensor;

(iii) contacting the probe, cell or sensor with the test sample; and

(iv) determining any change in FRET thereby to determine whether the test sample comprises the target substance;

use of a probe, cell or sensor of the invention, wherein the target binding site moiety of the probe, cell or sensor is capable of binding to a target substance, in the detection of the presence or absence of that target substance in a test sample;

a method for identifying an inhibitor of binding between two substances, which two substances would bind to each other in the absence of an inhibitor, comprising:

(i) providing a probe, cell or sensor of the invention, wherein the binding of the target binding site moiety of the probe, cell of sensor to the mimic moiety of the probe, cell or sensor mimics the binding of the two substances to each other;

(ii) determining the amount of FRET of the probe, cell or sensor;

(iii) contacting the probe, cell or sensor with a test substance; and

(iv) determining any change in FRET thereby to determine whether the test substance is an inhibitor of binding between the two substances; and

use of a probe, cell or sensor according to the invention, wherein the binding of the target binding site moiety of the probe, cell or sensor to the mimic moiety of the probe, cell or sensor mimics the binding of two substances to each other, in the identification of an inhibitor of binding between those two substances.

### BRIEF DESCRIPTION OF THE FIGURES

FIG. 1 shows the design and principle of operation of the probes.

A. In the absence of the target compound, the mimic moiety, for example a peptide/polypeptide, binds to the target binding site moiety, for example a peptide/polypeptide. The linker allows the two fluorophors to approach each other and a high level of FRET results.

B. The target molecule competes with the mimic moiety for the target binding site moiety causing separation of the two fluorophors and a decrease in FRET.

FIG. 2 shows the design of biotinylated probes.

A. The mimic moiety comprises a peptide sequence capable of biotinylation. Avidin binds to the biotinylated probe and the resulting complex can subsequently bind with a biotinylated substrate. A biotinylated oligonucleotide is shown bound to the target moiety, which is, for example, a transcription factor.

B. An inhibitor binds to the transcription factor, disrupting the binding between the oligonucleotide and the transcription factor. The distance between the two fluorescent peptides increases and FRET is thus reduced.

FIG. 3 shows the arrangement of the excitation source (a blue LED, blue laser or other appropriate light source) and two detectors around a flow-through cell containing pads of sensors to different compounds in a flow-through array detector which may be used in the present invention.

A. Cross section.

B. Surface view.

FIG. 4 shows a schematic map of the pTrcCFRET3 plasmid.

FIG. 5 sets out the sequence of the pTrcCFRET3 plasmid.

### DETAILED DESCRIPTION OF THE INVENTION

The probes of the invention comprise two fluorescent polypeptides connected by a linker. A target binding site moiety is attached to one of the fluorescent polypeptides. A mimic moiety is attached to the other fluorescent polypeptide. The mimic moiety has a three dimensional structure that is complementary to the structure of target binding site moiety. Thus, the mimic moiety can bind to the target binding site moiety.

The arrangement of the various domains of the probes is such that, typically, the target binding moiety and mimic moiety are free to interact with each other. When the target binding site and mimic moieties bind, the separation of the two fluorescent polypeptides (the fluorophors) is reduced and fluorescence resonance energy transfer FRET) occurs between the two fluorophors.

In the presence of a substance that disrupts the binding of the target binding site moiety to the mimic moiety, the separation between the target binding site moiety and mimic moiety may increase and consequently the separation between the fluorophors may also increase. As the separation of fluorophors increases, the level of FRET is reduced.

Probes of the invention may be designed so as to detect substantially any substance. In such probes, the target binding site moiety is capable of binding the substance, ie. the “target substance”, which the probe is designed to detect. The mimic moiety binds to the target binding site moiety in a way that mimics the binding of the target substance to the target binding site moiety.

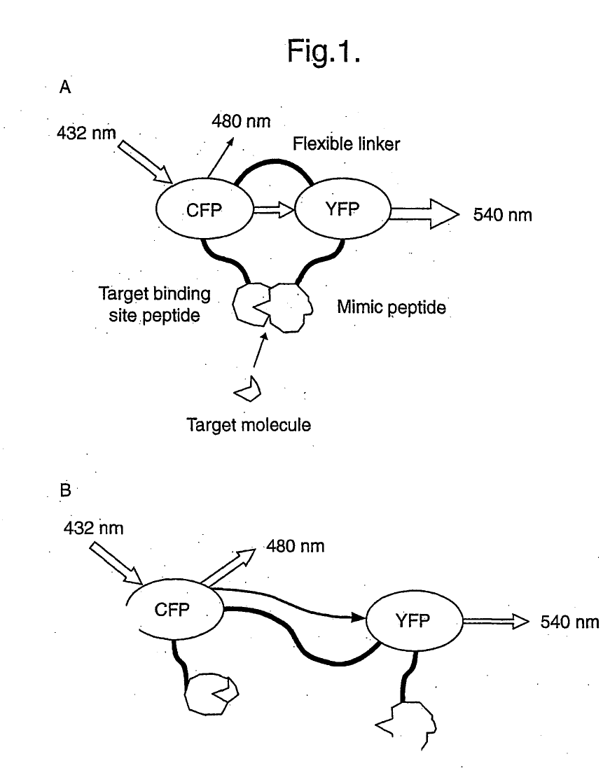

In the absence of the target substance, the target binding site and mimic moieties are free to bind with each other, the separation of the fluorescent polypeptides is reduced and FRET occurs (FIG. 1A). In the presence of the target substance, that substance will compete with the mimic moiety for binding with the target binding site moiety and displace the mimic moiety from the target binding site moiety. If the target substance displaces the mimic moiety, the separation between the target binding site and the mimic moieties increases, the separation of the fluorophors increases and the amount of FRET is thus reduced (FIG. 1B).

The degree of mimic moiety displacement increases as the amount of target substance increases and thus probes of the invention may provide a quantitative indication, as well as qualitative indication, of the amount of target substance.

Probes of the invention may also be designed to screen for inhibitors which are capable of disrupting, reducing or even preventing two substances from binding to each other, which two substances, in the absence of an inhibitor, would bind to each other. In such probes the target binding site moiety and mimic moiety are chosen such that the way in which they interact mimics the binding interaction of the two substances of interest.

In the absence of an inhibitor, the target binding site and mimic moieties of an appropriate probe are free to bind with each other. The separation of the fluorophors is reduced and FRET occurs. In the presence of an inhibitor the binding of the target binding site moiety and the mimic moiety is disrupted. The separation of the target binding site moiety and the mimic moiety increases and FRET is reduced.

Combinatorial libraries of chemicals, for example, may be screened to identify inhibitors within those libraries that can disrupt the binding of substantially any two substances that, in the absence of an inhibitor, will bind to each other.

Probes of the invention may also be designed to screen for stimulators, which increase or promote binding, between two substances. In such probes, the target binding site and mimic moieties are chosen such that they mimic the two substances of interest.

In the absence of the stimulator, the target binding site moiety and mimic moiety of an appropriate probe may bind to each other weakly or not at all. Thus, the separation of the fluorophors may be such that there is no FRET or FRET levels are low. In the presence of a stimulator, the target binding site and mimic moieties bind to each other or bind to each other more strongly than in the absence of the stimulator. The separation between the fluorophors is reduced and FRET is increased.

Thus, probes of the invention may be used to identify, for example, a factor which increases the strength of binding between two substances or, a factor whose presence is necessary for the binding of two substances to take place. Combinatorial libraries, for example, may be screened to identify stimulators and/or stabilisers of binding interactions.

In summary, a probe of the invention comprises five domains: a domain that binds the mimic moiety (the target binding site moiety), a domain that binds to the target binding site moiety (the mimic moiety), a donor fluorescent polypeptide, a linker and an acceptor fluorescent polypeptide. In an alternative version of the probe, the acceptor fluorescent polypeptide may be replaced by a non-fluorescent polypeptide, which has an absorption spectrum overlapping with that of the donor fluorescent polypeptide.

Typically the linker is a peptidelpolypeptide and is connected to the two fluorescent polypeptides by peptide bonds. Also, the target binding site and mimic moities are typically peptides/polypeptides and thus may be conveniently attached to their respective fluorescent polypeptides by peptide bonds. Thus, a probe of the invention is typically a single polypeptide. When a probe is a single polypeptide, polynucleotides may be obtained which encode that probe. Such polynucleotides can be used in the manufacture of probes by, for example, expression in bacteria or transcription and translation of the polynucleotides in cell-free systems.

The mimic moiety will typically be a peptide/polypeptide. However, the mimic moiety may: comprise non-peptide components; be connected to a non-peptide substance; or may comprise a peptide sequence which is capable of being connected to a non-protein substance. The non-peptide components may be, for example oligonucleotides or glycoconjugates.

A preferred probe of the invention comprises a peptide sequence capable of biotinylation, for example the mimic moiety may comprise such a sequence. A probe comprising a biotinylation target sequence can be biotinylated and subsequently bound to streptavidin. Addition of a biotinylated substrate to a probe-streptavidin complex gives rise to the formation of a probe-substrate complex. Typically, the target binding site moiety, which may be a peptidelpolypeptide, is capable of binding to the substrate and therefore such a probe may be used in detection of the substrate. The substrate may be, for example, a peptide, an oligo/polynucleotide, a carbohydrate, a lipid or other organic molecule.

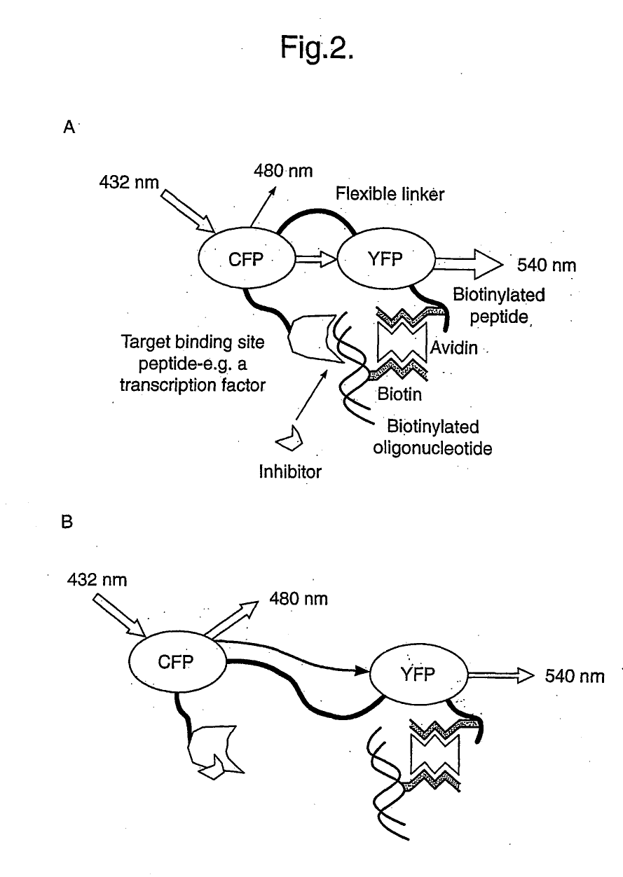

Such biotinylated probes provide a relatively straightforward route to the production of probes which can be used to detect the presence or absence of non-peptide components, including mRNA, DNA, carbohydrates, lipids and other organic molecules in test samples. Additionally, such probes may also be used to identify an inhibitor of binding between two substances. FIG. 2 shows how a biotinylated probe may be used to screen for an inhibitor of the binding interaction between a transcription factor and the nucleotide motif to which that transcription factor binds.

Biotinylated probes may produced by using a 17 residue biotin acceptor sequence that acts as a substrate for biotin ligase and permits the creation of endogenously biotinylated proteins. A suitable biotin acceptor sequence is MSGLNDIFEAQKIEWHE, which is based on the minimal acceptor sequence (Schatz, 1998, Biotechnology 11, 1138-1143) as adapted for higher, affinity (Beckett et al., 1999, Protein Sci. 8, 921-929). A polynucleotide construct encoding a probe, wherein the sequence encoding the mimic moiety comprises a nucleotide sequence encoding the biotinylation sequence, can be expressed in a bacterial strain over-expressing BirA (biotin ligase). This results in the expression of a protein which is biotinylated. The protein can be biotinylated at the N- or C-terminal end, depending on the location of the biotinylation peptide. This technology has been developed by Avidity under the trade name Avitag (U.S. Pat. No. 5,723,584).

Biotinylated probes can be purified on affinity columns comprising streptavidin bound to 2-imino-biotin attached to the column support. The probe-avidin complex is typically then released by dissociation from the 2-imino-biotin column support at low pH, for example pH4.0. The approach leads to a purified probe-streptavidin complex with a free binding site for biotin following release from the 2-imino-biotin column. Subsequent addition of any biotinylated substrate will allow reconstitution of complete probe.

Appropriate pairs of fluorescent polypeptides are those which exhibit FRET. That is, the donor polypeptide must be capable of absorbing light which excites an electron to a higher energy level. The electron will lose energy as it decays back to its ground state. The acceptor polypeptide must in turn be capable of accepting that energy to become excited itself. The extent to which FRET takes place is critically dependent on the overlap of the spectra of the fluorescent polypeptides and their separation. When selecting pairs of fluorescent polypeptides for use in a probe of the invention, various spectroscopic properties of the donor and acceptor need to be considered: (1) there needs to be sufficient separation in excitation spectra if the donor fluorescent polypeptide is to be stimulated selectively; (2) there needs to be an overlap between the emission spectrum of the donor and the excitation spectrum of the acceptor to obtain efficient energy transfer; and (3) reasonable separation in emission spectra between donor and acceptor fluorescent polypeptides is required to allow the fluorescence of each chromophore to be measured independently.

Suitable polypeptides include those from the green fluorescent protein (GFP) family of polypeptides, which are derived from the jellyfish species Aequoria victoria. Several basic classes of useful GFP mutants have been described, including: (1) red-shifted GFP, which has an emission peak most like that of wild-type GFP round 511 nm, but lacks the near-UV 395 nm excitation peak; (2) blue fluorescent protein (BFP); (3) cyan fluorescent protein (CFP); (4) sapphire; and (5) yellow fluorescent protein (YFP). For a review of GFPs see Pollok and Heim, 1999, TIBS 9, 57-60. Further GFP variants exist, for example a pH-insensitive CFP has been produced (Miyawaki et al. 1999, Proc. Natl. Acad. Sci. USA 96, 2135-2140) The coding sequences for these polypeptides are known and those polynucleotide sequences may be used to produce the corresponding polypeptides. Further suitable fluorescent proteins may be used which are derived from species other than Aequoria victoria.

Suitable pairs of GFPs include BFP (as donor) and red-shifted GFP, CFP and YFP and pH-insensitive CFP and YFP. Further combinations of GFPs and of other types of fluorescent proteins may be derived empirically.

In a probe of the invention, the second (acceptor) fluorescent polypeptide may be replaced by a non-fluorescent moiety, for example a non-fluorescent polypeptide. Suitable non-fluorescent polypeptides will have an absorption spectrum which overlaps with that of the first (donor) fluorescent polypeptide and will therefore be able to quench the fluorescence of the donor polypeptide.

A number of non-fluorescent polypeptides absorb strongly, including cytochromes, blue-light photoreceptors, heme proteins, phycobiliproteins, phytochromes and rhodopsins. Absorption by such polypeptides generally involves an attached prosthetic group or a conjugated metal ion.

In a further probe of the invention, the second (acceptor) fluorescent polypeptide may be replaced by a chemical dye attached to an immobilising surface (see below) and the mimic moiety coupled directly to a His₆ tag on the linker (see below) to lock it into close proximity with the surface.

FRET can be measured by any method known to those in the art including measurement of acceptor emission intensity, donor emission intensity or changes in donor emission lifetime.

Typically, FRET can be measured by monitoring changes in fluorescence intensity from the donor and acceptor, i.e. the ratio of emission of the two fluorescent proteins is recorded. For example, in the case of a probe comprising cyan fluorescent protein (CFP) and yellow fluorescent protein (YFP), excitation of CFP can be achieved by excitation with light at 430 to 440 nm. Some of the energy may be transferred to the YFP by FRET. This energy is emitted at a much longer wavelength (540 nm). Thus, such a probe should be monitored at both 480 nm and 540 nm.

Alternatively or in addition, resonance energy transfer may also be monitored by a change in the fluorescence lifetime of the donor fluorescence. Thus, measurement of the lifetime of the donor fluorescent polypeptide (and possibly also of the acceptor fluorescent polypeptide to improve sensitivity) may be recorded. This type of measurement is particularly useful in monitoring probes in which the acceptor fluorescent polypeptide is replaced by a non-fluorescent moiety.

Any suitable light source may be used to cause excitation of the donor fluorescent polypeptide, for example a xenon arc lamp, mercury arc lamp, tungsten-halogen lamp, laser or LED. Light emission from both the donor and acceptor fluorescent polypeptides may be measured by any suitable detector, for example a photomultiplier, a silicon-detector, a charge-coupled device (CCD) detector, diode array or diode arrays or a CCD-camera or by surface plasmon resonance. Particular wavelengths may be selected using for example interference filters, absorption filters, dichroic mirrors, prisms or diffraction gratings. The light sources, detectors and wavelength selectors may be combined in currently available instruments including fluorimeters, fluorescent plate readers, photometry systems, confocal microscopes, multiphoton microscopes and ratio imaging devices.

The two fluorescent polypeptides are connected by a linker. Typically, the linker is sufficiently flexible to allow the separation between the fluorescent polypeptides to vary. Thus, altering the flexibility of the linker will typically alter the apparent binding affinity of the target and mimic. Therefore the nature of the linker will be an important determinant of the sensitivity of a probe of the invention. The flexibility of the linker will be influenced by the length of the linker and the precise composition of the linker.

The linker, typically a peptide/polypeptide, comprises one or more of: (1) a sequence capable of being recognised and bound by an immobilized component; (2) a protease cleavage site; (3) a non-analyte binding site; (4) two or more copies of the sequence (SerGly₃); or (5) one or more copies of a rod domain from a structural protein.

A number of factors will affect the amount of fluorescence resonance energy transfer (FRET) for a probe constructed using a linker between two defined fluorescent proteins, such as CFP and YFP, including: (i) the separation of the two fluorophors, where the energy transfer is proportional to the sixth power of the distance between the donor and acceptor pair; (ii) the orientation factor between the donor and acceptor electric transition dipole moments; (iii) the quantum efficiency of the donor; and (iv) the integral of the spectral overlap of the absorption spectrum of the acceptor and the emission spectrum of the donor.

In the case of CFP and YFP, the quantum efficiency of the donor and the spectral overlap are pre-defined and are not expected to vary provided the environment is pH buffered. In contrast, changes in the amount of FRET, and hence the ability of the probe to report the presence of the analyte, can be achieved by varying either the separation distance of the two fluorescent proteins or their relative dipole orientation or both.

In addition to affecting the FRET signal, the linker will also affect the apparent binding constant between the target binding site and mimic moieties and the kinetics of the binding process. These will also be functions of the length of the linker, the flexibility of the linker and stearic constraints that are imposed on the orientation of the target binding site and mimic moieties.

The design of a probe of the invention therefore encompasses a family of linkers in which these properties may be systematically varied by, for example, inclusion of unique restriction sites within a nucleic acid coding for the polypeptide, allowing multiple insertions of distinct motifs.

(i) Flexibility in the linker may be achieved by the use of a (SerGly₃)₄ motif and/or hinge sequences from heavy chains of antibodies in a linker of a probe of the invention. Such motifs may be used singly or multiple copies may be used (i.e. a copy number of greater than one may be used).

Such motifs are present at a copy number of one or more, or two or more, for example from 2 to 10, preferably from 2 to 6, more preferably from 2 to 4 copies. The multiple copies of the (SerGly₃)₄ motif and/or hinge sequences may be arranged end to end as a tandem array or may be separated by other sequences. In the latter case, the (SerGly₃)₄ motifs and/or hinge sequences may flank other components of the linker, for example a His tag, an epitope tag and/or a cleavage site (see below).

(ii) Rigidity may be achieved by incorporation of rod domains from structural proteins, such as collagen. The length of these rigid segments can be varied from, for example, 10 to 100 amino acid residues, preferably from 20 to 60 amino acid residues. The probe may contain more than one rod domain, for example from 1 to 10 domains, preferably from 1 to 6 domains, more preferably from 1 to 4 domains. Identical or different domains may be used within one linker. Again, the rod domains may be arranged end to end in tandem or may be separated by other sequences.

(iii) Attachment motifs useful in immobilisation and/or purification may be included in a linker of a probe of the invention. This allows facile purification of a probe from a suspension culture of bacteria harbouring a plasmid encoding a probe of the invention. It also allows immobilisation of a probe in the wells of for example a microtitre plate. Immobilisation may be useful as a mechanism for controlling undesirable (i.e. target substance independent) FRET due to intermolecular dimerisation of the fluorescent polypeptides of a probe. Immobilisation may also serve to limit through-chain energy transfer, which would itself limit the useful FRET ratio change with target substance binding.

Thus, a probe of the invention may comprise a peptide sequence capable of being recognised and bound by an immobilised component. This would preferably be a hexa-histidine tag (His₆), an antibody epitope, or a sequence recognised by a protein modification enzyme (for example a biotinylation site, glycosylation site or a phosphorylation site).

Such sites may be used in the preparation of a purified recombinant fusion protein (i.e. a probe of the invention) from a complex mixture (e.g. a bacterial lysate), by transiently immobilising it to a surface such as a bead in a column. In addition, immobilisation of a probe through this sequence can be used to anchor the probe to a surface within a detection instrument, both facilitating construction of an instrument containing the probe and also restricting unwanted dimerisation and target substance-independent intermolecular FRET signals that might occur with free probe in solution.

More than one attachment site may be present in a linker of a probe of the invention, for example 2, 3 or 4 attachment sites. The multiple attachment sites may be the same or, more typically, different.

The orientation and restriction on movement of the fluorescent proteins will be affected by the number and order of elements (i), (ii) and (iii) incorporated into a linker of a probe of the invention. A linker may be modified further by the addition of discrete amino acid residues, such as proline, to twist the amino-acid chain.

The number and order of each of the domains described above may be varied. Examples of typical combinations include:

(a) target binding site moiety-CFP-(SerGly₃)ₙ-attachment domain-(SerGly₃)ₘ-YFP-mimic moiety

where: n is may be from 0 to 4, m may be from 0 to 4 and n+m may be from 2 to 4; or n or m each independently may be 4, 8, 12, 16, 20, 24 or 28.

(b) target binding site moiety-CFP-(SerGly₃)ₙ-rod domain-attachment domain-(SerGly₃)ₘ-YFP-mimic moiety

where n and m are as above.

(c) target binding site moiety-CFP-rod domain-(SerGly₃)ₙ-rod domain-YFP-mimic moiety

where n is as above.

(d) target binding moiety-CFP-prolineₚ-rod domainᵣ-(SerGly₃)_{q}-rod domainₗ YFP-mimic moiety

wherein p is from 0 to 4, q is from 0 to 4, p+q is from 1 to 4 and r is from 1 to 5. Alternatively, q may be 4, 8, 12, 16, 20, 24 or 28.

The linker also provides a convenient position within the probe of the invention to incorporate functional sites distinct from those associated with detection of the target substance.

Thus, a protease cleavage site or sites may be incorporated into a linker of a probe of the invention. The cleavage site may be for any type of protease, such as enterokinase or Factor X. More than one site may be included in a linker, for example 2, which typically will be different. Cleavage of a probe on a substrate might ensure stoichiometric immobilisation of appropriately positioned donor and acceptor fluorescent polypeptide components so that they lack a covalent linkage. This may offer a useful lowering of the spontaneous FRET background by reducing through-chain energy transfer. Thus, post-immobilisation cleavage of a probe (in the form of a polypeptide) will be useful in lowering background FRET.

In addition, cleavage of a probe will permit the complete separation of the donor and acceptor fluorescent polypeptides, which will in turn allow the minimum level of FRET in the system to be determined.

Alternatively or in addition, a probe of the invention destined for expression within living cells may incorporate a non-analyte (target substance) binding site within the linker to confer, for example, sub-cellular localisation of the probe to specific cellular structures. This might, for example, allow the probe to be tethered to the plasma membrane or nuclear envelope to report localised analyte concentrations in specific region of the cell. Such a non-analyte binding site might be used to ensure appropriate subcellular localisation of a probe within living cells that are themselves used as a tool to measure particular analytes by virtue of the ability of the cellular machinery to selectively internalise and concentrate said analyte.

Alternatively, probes incorporating a non-analyte binding site might act as indirect sensors of certain molecules that interact with a signalling system within the cell that impinges on the analyte targeted by the sensor.

Targeting to other organelles rather than regions within the cytoplasm may have to be carried out differently, as typically the targeting information resides on the C- or N-terminus of the protein rather than within the polypeptide itself. In the configuration envisaged for probes of the invention, this would require modification to either the target binding site moiety or the mimic moiety and would thus fall outside of properties of the linker.

A preferred probe will have therefore have the overall structure:

target binding site moiety (a peptide)-CFP-(SerGly₃)ₙ-His₆-EK-antibody epitope-(SerGly₃)ₘ-YFP-mimic moiety (a peptide)

where n or m each independently may be 1, 4 or 8.

In a probe of the invention, which of the fluorescent polypeptides (donor or acceptor) is attached to the target binding site moiety or the mimic moiety is generally immaterial. When the donor is attached to the target binding site moiety the acceptor is attached to the mimic moiety and vice versa. Typically, the target binding site moiety and the mimic moiety are both peptides/polypeptides and are attached to their respective fluorescent polypeptides by peptide bonds. However, the target binding site and mimic moieties may be attached to the fluorescent polypeptides by other types of non-peptide bond connection.

Probes may comprise polypeptides which have been post-translationally modified. Thus, probes may comprise post-translational modifications such as phosphorylation, fatty acyl modification (including farnesylation, geranylgeranylation or palmitoylation) or glycosylation. Probes may comprise more than one type of post-translational modification and may comprise up to 10, up to 20, up to 30, up-to 50, up to 100 or more than 100 post-translational modifications.

Typically, a probe will comprise a single polypeptide and therefore a probe may be encoded by a single polynucleotide. The isolation of appropriate target binding site and mimic peptides and the corresponding polynucleotides which encode those peptides allows the construction of polynucleotides encoding probes. Thus, the invention also provides polynucleotides which encode probes of the invention.

The invention further provides double stranded polynucleotides comprising a polynucleotide of the invention and its complement. Polynucleotides of the invention may comprise DNA or RNA. They may also be polynucleotides which include within them synthetic or modified nucleotides. A number of different types of modification to polynucleotides are known in the art. Such modifications may be carried out in order to enhance the in vivo activity, lifespan, nuclease resistance or ability to enter cells of polynucleotides of the invention. For example, phosphorothioate oligonucleotides may be used. Other deoxynucleotide analogs include methylphosphoniates, phosphoramidates, phosphorodithioates, N3′P5′-phosphoramidates and oligoribonucleotide phosphorothioates and their 2′-O-alkyl analogs and 2′-O-methylribonucleotide methylphosphonates.

Alternatively mixed backbone oligonucleotides (MBOs) may be used. MBOs contain segments of phosphothioate oligodeoxynucleotides and appropriately placed segments of modified oligodeoxy- or oligoribonucleotides. MBOs have segments of phosphorothioate linkages and other segments of other modified oligonucleotides, such as methylphosphonate, which is non-ionic, and very resistant to nucleases or 2′-O-alkylohlgoribonucleotides.

Polynucleotides such as a DNA polynucleotide according to the invention may be produced recombinantly, synthetically, or by any means available to those of skill in the art. They may also be cloned by standard techniques. The polynucleotides are typically provided in isolated and/or purified form. Although in general such techniques are well known in the art, reference may be made in particular to Sambrook et al., 1989, Molecular Cloning: A Laboratory Manual.

Polynucleotides of the invention can be incorporated into a recombinant replicable vector. The vector may be used to replicate the nucleic acid in a compatible host cell. Thus, in a further embodiment, the invention provides a method of making probes of the invention by introducing a polynucleotide of the invention into a replicable vector, introducing the vector into a compatible host cell, and growing the host cell under conditions which bring about replication of the vector. The vector may be recovered from the host cell.

Preferably, a polynucleotide of the invention in a vector is operably linked to control sequences which are capable of providing for the expression of that polynucleotide by the host cell, i.e. the vector is an expression vector. The term “operably linked” refers to a juxtaposition wherein the components described are in a relationship permitting them to function in their intended manner. Regulatory sequences, such as promoters and terminators, “operably linked” to a polynucleotide are positioned in such a way that expression of the polynucleotide is achieved under conditions compatible with the regulatory sequences. Typically regulatory sequences will comprise a promoter (generally positioned 5′ to the polynucleotide), and/or a terminator and/or translation initiation sequence (eg. GCCACCATGG or GCCCCCATGG) and/or a translational stop codon (eg. TAA, TAG or TGA) and/or polyadenylation signal and/or one or more enhancer sequences and/or RNA pause site. The control sequences may increase transcription and or translation of the polynucleotide or may direct expression of the polynucleotide only in certain tissues.

The vectors may be, for example, plasmid, cosmid, virus or phage vectors provided with an origin of replication, and optionally any of the control sequences described above. The vectors may contain one or mote selectable marker genes, for example an ampicillin resistence gene may be used with a bacterial plasmid and a kanamycin resistance gene may be used with a plant vector.

Vectors may be used in vitro, for example for the production of RNA or introduced into a host cell. Any transfection or transformation technique may be performed in order to introduce a vector into a cell, for example, electroporation, salt precipitation, liposome mediated, protoplast fusion, viral infection, microinjection or ballistics techniques. The introduction may be aided by a natural mechanism by which the cell can take up material, such as pinocytosis or phagocytosis.

Thus, a further embodiment of the invention provides a host cell harbouring a vector of the invention. Cells transformed or transfected with vectors of the invention may allow for the replication and/or expression of polynucleotides encoding probes of the invention. Therefore, this invention also provides a cell harbouring a probe of the invention. The cell may be present in a culture of cells which culture also comprises a medium capable of supporting the cells.

The cells will be chosen to be compatible with the said vector and may be prokaryotic, such as a bacterial cell (eg. E. coli) or eukaryotic such as yeast, fungal, insect, plant, animal, for example, mammalian or human cells. The cells may be undifferentiated or differentiated. The vector may exist in an episomal state in the host cell or the polynucleotide incorporated into the vector may become integrated into the genome of the cell.

Promoters and other control sequences may be selected to be compatible with the host cell for which expression is desired. For example, yeast promoters include S. cerevisiae GAL4 and ADH promoters, S. pombe nmt1 and adh promoter. Plant promoters include the CAMV 35S and rubisco ssu promoters and mammalian promoters include the metallothionein promoter which can be induced in response to heavy metals such as cadmium. Viral promoters such as the SV40 large T antigen promoter or adenovirus promoters may also be used for expression in mammals. All these promoters are readily available in the art.

The cell can be used in an expression system to produce the gene product. A preferred expression system is the baculovirus system. Thus, in a further aspect the invention provides a process for preparing a probe according to the invention, which comprises cultivating a host cell transformed or transfected with an expression vector incorporating a polynucleotide encoding the probe under conditions which allow expression of the probe, and optionally recovering the expressed probe.

Probes of the invention can be designed to detect substantially any target substance. The target substance may be any substance for which an appropriate target binding site moiety-mimic moiety pair can be generated.

Probes may also be designed to identify an inhibitor of binding between two substances, which two substances would bind to each other in the absence of an inhibitor. Substantially any binding interaction between two substances can be screened, providing that a target binding site moiety and mimic moiety pair can be generated, the binding to each other of which mimics the binding of the two substances of interest to each other.

Appropriate target binding site moieties may be isolated by any method and suitable methods will be well known to those skilled in the art. For example, antibodies specific for a particular target substance or specific to one of a pair of substances, whose binding to each other is to be investigated, may be isolated. This will allow the isolation of the coding sequences for appropriate single chain antibodies. Thus, it may be convenient to use antibodies from organisms that produce single chain antibodies, for example camels.

An alternative strategy for isolating target binding site moieties is to select sequences of proteins or protein motifs which have a defined substrate specificity. Also, sequences of proteins or protein motifs that are glycosylated may provide suitable target binding site moieties for sugars and carbohydrates.

Similarly, mimic moieties may be isolated by any method and suitable methods will be well known to those skilled in the art. For example anti-idiotypic antibodies to the target substance may be generated and thus the coding sequences for appropriate single chain anti-idiotypic antibodies. Alternatively, mimic moieties may be generated by selecting sequences of proteins or protein motifs which have a defined substrate specificity.

A probe may be used to detect the presence or absence of a ligand by the use of an idiotype network. In this approach a pair of monoclonal antibodies is used, one of which is an internal image anti-idiotype of the other. The method then requires the expression of each antibody as an ScFv, one as the target binding site moiety and the other as the mimic moiety. This gives rise to binding of the target binding site moiety to the mimic moiety and therefore a FRET signal in the resting state. The probe can then be used to specifically identify the original ligand (of which the anti-idiotype is an internal image) by a competition effect resulting in the loss of the FRET signal. This approach is thus a general approach for the detection and measurement of any ligand with the specificity of the starting antibodies. It will be clear that the ligand does not necessarily have to be a peptide/protein. The ligand can be any substance for which the necessary pair of monoclonal antibodies can be generated.

Also, a combinatorial library of peptide sequences may be screened for binding to a target binding site moiety. Additionally a library of polynucleotides encoding probes could be produced, wherein the mimic moiety domain is encoded by a library of polynucleotides. The library can be screened for FRET. Clones showing high levels of FRET should comprise polynucleotides which encode a mimic moiety which binds to the target-site moiety.

In the above techniques antibodies may be isolated by any suitable technique. For example, the target substance may be used to immunize an animal such as a rabbit, rat, mouse or chicken. Alternatively, expression libraries or phage display libraries may be screened. Those technique allow the convenient recovery of polynucleotides encoding suitable antibodies.

When a probe of the invention is used to detect the presence or absence of a particular target substance, the specificity of a probe will depend on the specificity of the target binding site moiety for the target substance and on the specificity of the mimic moiety. The sensitivity of the probe will depend on the dissociation constant of the target binding site moiety, the dissociation constant of the mimic moiety-target binding site moiety interaction and the strain/flexibility imposed by the linker. Changing any one or more of these parameters should result in probes with a spread of specificities and sensitivities.

Typically, a probe will be specific for one target substance or a small number, for example 2, 3, 5 or 10 target substances. However, the invention also provides probes which are less specific and thus may be capable of detecting a family of substances, for example, at least 10, at least 20, or at least 50 substances. The family of substances will typically share similarity in an aspect of their structure.

Probes with high sensitivity are preferred. Changes in FRET are typically measured as changes in the ratio of donor fluorescence to acceptor fluorescence, although changes in donor emission lifetime can also be used. A reduction is FRET is typically indicated by an increase in the ratio of donor fluorescence to acceptor fluorescence. The greater the increase in the ratio of donor fluorescence to acceptor fluorescence on binding a target substance species, the greater the sensitivity of the probe. Preferred probes are those which, when one probe molecule binds one target substance species, exhibit a reduction in FRET and thus an increase in the ratio of donor fluorescence to acceptor fluorescence of from 1.5 to 2.0, preferably from 0.5 to 3.0 or more preferably from 0.1 to 5.0.

New probes can be screened to establish whether they exhibit FRET. Probes that exhibit FRET may be titrated with the target substances to determine the sensitivity. Probes may then be screened with substances related to the target substances to determine the probe specificity.

Probes of the invention may be used to detect a target substance in any test sample. Thus this invention also provides a method for detecting the presence or absence of a substance in a test sample. Typically, the test sample will be a fluid. Probes are typically used as substantially purified proteins. Alternatively, living cells, for example bacterial cells, that express a probe (or probes) may be used.

Typically, a method for detecting the presence or absence of a substance comprises: determining the amount of FRET from a probe, a cell harbouring a probe or a sensor comprising a probe; contacting the probe, cell or sensor with a test sample; and determining any change in FRET from the probe, cell or sensor, thereby to determine the presence or absence of the target substance in the test sample. As well as giving a qualitative indication of the presence or absence of a target substance, the method of the invention may also provide a quantitative measurement of the amount of the substance present in the test sample.

Probes of the invention may be used singly or in combination. Thus, two or more, for example, three, five, ten or more, may be used simultaneously in a method of the invention for detecting the presence or absence of a test sample. Typically, if more than one probe of the invention is used simultaneously, the donor and acceptor polypeptides of each probe will be different. When more than one probe is used, preferably any probe used will not interfere with the ability of another probe to undergo FRET. FRET from each probe can be measured sequentially or simultaneously, using appropriate detection apparatus. The use of more than one probe in a method of the invention for detecting the presence or absence of a substance will allow the presence or absence of more than one substance in a test sample to be determined.

When using a substantially purified probe, any suitable technique may be used to detect the presence or absence of a test sample. One of the following two approaches is typically used:

(1) Equilibrium approach—a probe which has an affinity comparable to the typical concentration of the target substance is used, but at a comparatively low absolute amount. In this way, only a small proportion of the population of target substance molecules binds the probe molecules and thus the concentration of the target substance is not markedly affected.

Calibration of the probe may be carried out using media comprising known amounts of the target substance. Suitable controls may be used, for example media in which the target substance is not present may be used. Also, competition experiments between the unknown and known amounts of the target substance may be carried out to test for interference by other compounds present in the test sample. Additionally, the probe may be washed out after a test and recalibrated to test for irreversible modification of the probe.

(2) Saturation (affinity) approach—the probe has a very high affinity for the target substance in comparison to the concentration of the target substance and is present in amounts such that the test sample is substantially depleted in respect of the target substance. This approach maybe used in static systems, whereby the probe is placed in contact with a known volume of the sample or in a flow-through system whereby a solution of the test sample and/or controls are passed over the immobilized probe. Similar controls may be used to those described for (1) above. In addition, when a sensor comprising immobilized probe is used, the profile of binding along the length of the sensor can also be monitored and analysed to calculate the binding affinity of the target and probe.

When a probe is used to detect the presence or absence of a substance and that probe is harboured by a cell, appropriate assay methods may be more complex. Calibration of the probe is preferred where known concentrations of the target have somehow been introduced into the cell. For ions, this may be carried out through the use of ionophores. For organic molecules, a non-specific permeabilisation agent, for example streptolysin, may be used in a medium containing known amounts of the target substance. Alternatively, the calibration is based on the response of a probe determined in a medium designed to mimic the environment of the probe within the cell.

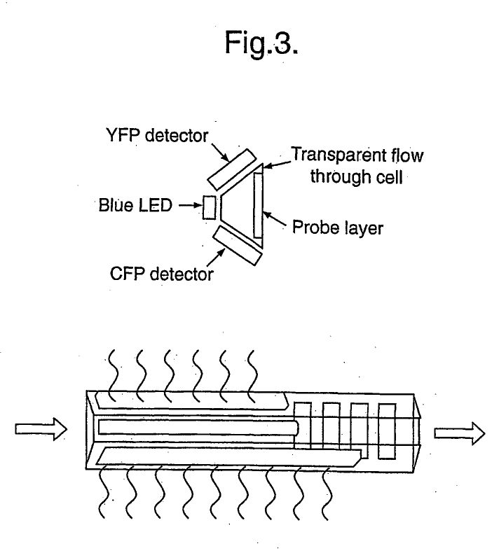

Probes of the invention may be incorporated into a sensor. Preferably, such a sensor is small and portable. Thus the present invention also provides a sensor comprising a probe of the invention, a light source which is capable of exciting the probe and a detector which is capable of measuring the amount of FRET. A typical sensor is illustrated in FIG. 3. The sensor is generally based on silicon chips with five modules per probe: (i) a blue-light emitting diode or small blue laser, or an LED or small laser of a different wavelength if the donor fluorescent polypeptide responds to a different wavelength of light; (ii) a pad for immobilising the probe, accessible to (iii) a sample delivery/flow-through system; (iv) a first silicon detector; and (v) a second silicon detector, wherein the two silicon detectors have different spectral sensitivities to measure the fluorescence from the two fluorescent polypeptides of the probe.

A typical low-cost detector comprises two silicon devices equipped with interference filters or coloured-glass filters with an appropriate peak transmission and bandwidth. Such detectors can be used singly or optionally can be arranged in arrays. Cooling to −20° C. may be required in some situations to achieve a good signal-to-noise ratio. A more complex system comprises a diode-array detector preceded by a prism or diffraction grating so that a complete emission spectrum can be collected, rather than just two emission wavelengths. Complete emission spectra may contain more information about whether the change in signal is entirely due to changes in FRET.

A sensor may also be in the form of a dual-multiplier system with light separated into two channels using a dichroic mirror and each channel equipped with an appropriate filter. Alternatively, the two photomultipliers with appropriate filters could be placed adjacent to the sample chamber as indicated for the silicon detector system, described above.

For remote applications, where complex electronics are not possible or undesirable, sensors based on film or phosphor imager plates would be suitable.

To minimize the amount of direct illumination received by the detectors, the detectors should each generally not be oriented at 180° C. or near 0° C. to the light source. Typically, for a probe immobilised on an opaque light substrate the detectors should be at an angle of about 45° C. to the light source. The sensor may comprise a flow-through cell with an array, typically parallel, of different probes so that the, presence of numerous target substances can be determined simultaneously. Additionally a flow-through cell may comprise a series/array of probes specific for the same substance may be used which differ in affinity for the target substance because, for example, the probes have linkers with different flexibilities.

Probes of the invention may be used to detect the presence of a substance, for example a metabolite, hormone, drug, toxin or pollutant in an extract, for example a fluid sample derived from any organism, including an animal or human, plant, fungus or microbe.

For example, a probe of the invention may be used to detect sugars, oligosaccharides or non-carbohydrate mimetics. For such use, the target binding site moiety of the probe comprises a recombinant monomeric plant lectin of the desired oligosaccharide binding specificity. The mimic peptide is endogenously biotinylated. The probe is activated by the attachment of a small biotinylated glycoprotein, to generate an interaction between the lectin and the cognate oligosaccharide recognition element with the consequent appearance of a FRET signal. Sugar, oligosaccharide or non-carbohydrate mimetics may then be detected by their ability to reduce this FRET signal.

A probe of the invention may also be used to determine the presence or absence of steroid hormones. Such an application makes use of the change in binding affinity of a sythetic peptide probe to a steroid hormone receptor, for example the estrogen receptor (ER) upon binding of a specific steroid hormone (Paige et al., 1999, Proc. Natl. Acad. Sci. USA 96, 3999-4004). The target binding site of a suitable probe may comprise sequence encoded by a cDNA corresponding to the estrogen receptor. The mimic moiety comprises one of a least three sequences:

(i) SSNHQSSRLIELLSR (this sequence shows no binding to ER except in the presence of estradiol);

(ii) SAPRATISHYLMGG (this sequence binds ER in the absence of steroids, but is released by estradiol or tamoxifen); or

(iii) SSPGSREWFKDMLSR (this sequence shows no binding to ER except in the presence of tamoxifen.

For sequences (i) and (ii), the probe operates in the opposite manner to that generally described above. That is, the target binding site and mimic moieties do not freely bind each other in the absence of the target substance. Rather, only in the presence of the target substance do the target binding site moiety and mimic moiety bind. Thus, in such cases the presence of the target substance will lead to a reduction in the separation of the fluorophors and therefore to an increase in FRET. In the description of probes above, typically the presence of the target substance is indicated by a decrease in FRET.

In the case of an animal or human, the sample could be, for example, blood, saliva, tears, cerebro-spinal fluid or semen. A probe may be used to determine the presence or absence of a particular substance in an animal or human sample. The presence of a particular substance in a substance may be indicative of a disease state. Alternatively, the absence of a particular substance may be indicative of a disease/clinical condition. Thus the invention provides a probe for use in a method of diagnosis practised on the human or animal body.

The invention also provides a method of diagnosis comprising determining the amount of FRET from a probe and then contacting an animal or human sample with a probe of the invention. Any change in FRET is determined and thereby the presence or absence of a particular target substance is determined. The disease state, healthy or otherwise, of the animal or human may thus be determined. The method is typically carried out ex vivo, ie. on a sample withdrawn from the subject.

Other applications in animals or humans include drug and alcohol testing and testing for exposure to toxins or pollutants.

A probe of the invention may also be used to detect air-borne substances, for example, atmospheric pollutants, if these substances are soluble. Thus, a probe of the invention can be provided in an aqueous medium which is exposed to the surrounding atmosphere. Any substances in the surrounding air which are soluble will dissolve in the probe containing medium and can be detected by a suitable probe or probes in the medium.

Probes of the invention may also be used to detect specific substances in plant, fungal or microbial, for example bacterial, extracts. Plant extracts, for example exudates, may be useful in determining the presence of plant pathogenic viruses or bacteria in a plant. Additionally, probes of the invention may be used to determine the presence and amount of trace elements or pollutants in plant extracts. Thus results of such assays may provide indirect measurements of soil quality and in some cases be indicative of particular types of soil pollution.

A further application of probes of the invention is to use them to detect proteins expressed in transgenic plants, or transgenic animals, fungi or microbes. When transgenic organisms are produced, often large numbers of so-called primary transformants have to be screened for expression of the transgene. Typically, time-consuming RNA and protein blotting techniques are used. Probes of the invention could be used to assay crude extracts in a more quantitative fashion that RNA and protein blotting and also more quickly than those techniques.

Probes may be used to detect for example contaminants or pollutants in for example, water supplies, soil or factory effluents. Probes may be used in quality control situations to detect substances, for example contaminants, in foodstuffs and medicaments.

This invention also provides multicellular organisms or parts thereof comprising a probe, polynucledtide, vector or cell of the invention. Typically such organisms will comprise a polynucleotide of the invention, such that the probe for which that polynucleotide codes is expressed in that organism or part thereof. In other words, the organisms or parts thereof may be transgenic for a polynucleotide of the invention. An organism or part thereof may comprise more than one polynucleotide, vector, cell or probe of the invention.

The expression of a probe may be constitutive or tissue specific and may persist for the whole of the organisms life-cycle or may be expressed at a particular developmental stage of the life-cycle. Different probes may be expressed at different times during the life-cycle of the organism. Thus organisms may be produced, wherein the probe is expressed under the control of a constitutive promoter or under the control of a promoter which directs spatially or temporally restricted expression. Suitable promoters are well known to those skilled in the art.

Any multicellular organism may comprise a probe, nucleotide, vector or cell or the invention, for example, fungi, plants and animals. Suitable plants may be monocotyledonous or dicotyledonous. Preferred monocots are graminaceous plants such as wheat, maize, rice, oats, barley and rye, sorghum, triticale and sugar cane. Preferred dicotyledonous crop plants include tomato, potato, sugarbeet and other beet crops; cruciferous crops, including oilseed rape; linseed; tobacco; sunflower, fibre crops such as cotton; and leguminous crops such as peas, beans, especially soybean, and alfalfa. Suitable animals include insects, for example the dipteran Drosophila melanogaster and mammals, for example mice, sheep, pigs or cows.

Multicellular organisms comprising probes, polynucleotides, vectors or cells of the invention may be generated according to techniques well-known to those skilled in the art. Generally, a polynucleotide of the invention is incorporated into a vector and that vector is used to transform or transfect a cell of the organism. That cell is then used to regenerate a multicellular organism, which will generally be able to replicate. Thus, the invention also provides a method of producing a transgenic organism which comprises transforming or transfecting a single cell of that organism with a polynucleotide of the invention and allowing that cell to develop into a multicellular organism.

Use of probes of the invention in living cells falls into two main classes: (i) use in isolated cells in culture; and (ii) use in intact multicellular organisms.

Isolated cells in culture may be microbial, for example bacterial, fungal, plant or animal, for example mammalian cells, which comprise a probe or probes of the invention. The probe or probes could be to any substance or substances including: metabolites, for example glucose, sucrose and NADPH; signalling molecules, for example Ca²⁺, H⁺, Ins(1,4,5)P3, cAMP, cGMO, testosterone; xenobiotics, for example toxins, drugs, metabolites of drugs (both prescription medications and drugs of abuse), herbicides, pesticides or fungicides; peptides such as calmodulin or knases; post-translational modification sites, for example phosphorylation, glycosylation or fatty acyl modification sites.

Cells containing probes may be grown in suitable media in, for example, multi-well plates or microscope chambers. Changes in FRET may be recorded using, for example, a fluorimeter, fluorescent plate reader, camera imaging system, confocal microscope or multi-photon microscope.

Assays may be for the indirect effects of a drug on the metabolism or physiology of a cell, rather than as a direct probe for the presence of a drug. Such systems can form the basis of high-throughput physiological screening systems. For example, in the case of a therapeutic drug which as well as having a therapeutic effect has unwanted side-effects, substances could be screened for their ability to reduce the side-effect. A probe is used which is specific for a physiological indication of the side-effect, for example, increased accumulation of a particular metabolite. Collections of substances, for example combinatorial libraries, could be screened for in high-throughput assays for substances which prevent increase of the metabolite and thus have the potential to ameliorate side-effects of the drug.

In multicellular systems the approach is similar to that outlined above for single cells, however, typically only the surface cell layers of a multicellular organism are accessible to non-invasive fluorescence techniques. Global fluorescence measurements can be made using photometry, fluorimetry, camera, confocal or multi-photon techniques. Tissue, cell or organelle specificity can be achieved using tissue-specific, developmental-specific and/or targetted probes, ie. probes that are expressed under the control of tissue- or developmental-specific probes or probes that comprise a targetting peptide.

For example, to determine changes in the plant hormone abscisic acid in the stomatal guard cells of a leaf, a probe directed to abscisic acid is expressed only in those cells. Changes in FRET of the probe are monitored using a non-imaging system. Alternatively, the probe is expressed constitutively throughout the plant, in which case measurements are made only from the guard cells using an imaging technique.

Probes of the invention may also be used to investigate binding between two substances, which two substances would typically bind to each other. Thus, the invention also provides a method for identifying an inhibitor of binding between two substances, which two substances would bind to each other in the absence of an inhibitor.

The types of binding interaction that may be investigated may be, for example, peptide-peptide interactions, peptide-carbohydrate, peptide-nucleic acid or peptide-ligand interactions. If a target binding site moiety and mimic moiety pair can be can identified, the interaction of which mimics the binding interaction of the two substances of interest, the interaction between those two substances can be investigated.

Typically, such methods will be used to investigate interactions which are of significance in human or animal disease states. For example, host recognition by a pathogen is often a critical step in infection. Probes of the invention may be used to investigate that pathogen-host recognition interaction. For example, some pathogens recognise carbohydrate species on the surface of host cells. An appropriate probe may be designed which can be used to identify inhibitors of the binding interaction between a pathogen and a carbohydrate molecule on the surface of a host cell. An inhibitor so identified may be used to disrupt the recognition interaction between the host and pathogen and therefore may be used to prevent infection of the host by the pathogen.

Thus, inhibitors identified by a probe of the invention may be used in a method of treatment of the human or animal body by therapy.

Probes of the invention may be designed for use in identifying inhibitors of estrogen stimulated transcription. Such a probe comprises the estrogen receptor (ER) as the target binding site moiety and is biotinylated at the mimic moiety. Therefore a biotinylated oligonucleotide bearing the estrogen receptor response element (ERE) can be attached to the mimic moiety. The ER will bind to the ERE and give a FRET signal unless an inhibitor of the DNA-protein interaction is present, in which case the FRET signal will be lost. Therefore such a probe could be used to screen for inhibitors of the growth of estrogen-sensitive breast tumors. Such a screen could be used to identify anti-tumor agents that act at a site distinct from that targeted by the synthetic estrogen, tamoxifen.

Probes of the invention can also be use to identify protease inhibitors. The target binding site moiety of a suitable probe comprises a recombinant protease and the mimic moiety comprises a known peptide inhibitor. Thus FRET is detected in the resting state as the inhibitor binds in the protease active site. The probe can thus be used to screen for active binding site inhibitors of the protease.

Probes of the invention may further be used to identify intracellular G protein signal inhibitors. Thus, probes can be used to identify novel classes of signal transduction inhibitor. In a suitable probe, the target binding site moiety comprises the cytoplasmic loop of a selected seven transrembrane receptor and the other end comprises the C terminal part of an alpha subunit of a heterotrimeric G protein complex. Since the C terminal region of the alpha subunit contains the receptor binding site and is functional in isolation, the probe displays FRET in the resting state. An inhibitor of this interaction would reduce the FRET signal.

Typically, a method for identifying an inhibitor of a binding interaction between two substances may be carried out by determining the amount of FRET from a suitable probe (or cell or sensor comprising such a probe) in the absence of a test substance; contacting the probe (or cell or sensor) with a test substance; and determining the FRET from the probe (or cell or sensor) thereby to determine whether the test substance can inhibit the binding interaction between the two substances of interest. Inhibition of the binding interaction will typically be indicated as a reduction of FRET of the probe (or cellor sensor). A suitable probe for use in such a method is one in which the binding of the target binding site moiety of the probe to the mimic moiety of the probe mimics the binding of the two substances of interest to each other.

Suitable control experiments can be carried out. For example, a candidate inhibitor can be tested with other probes of the invention, to determine that it specifically inhibits the interaction under investigation and is not simply a general, non-specific inhibitor of many binding interactions.

Any suitable format can be used for carrying out a method for identifying an inhibitor of a binding interaction. However, the screening method is preferably carried out in a single medium, most preferably in a single well of a plastics microtitre plate. Thus the method can be adapted for use in high though-put screening techniques.

Suitable test substances for inhibitors of binding interactions include combinatorial libraries, defined chemical entities, peptides and peptide mimetics, oligonucleotides and natural product libraries. The test substances may be used in an initial screen of, for example, ten substances per reaction, and the substances of batches which show inhibition tested individually. Furthermore, antibody products (for example, monoclonal and polyclonal antibodies, single chain antibodies, chimaeric antibodies and CDR-grafted antibodies) maybe used.

The following Example illustrates the invention:

### EXAMPLE

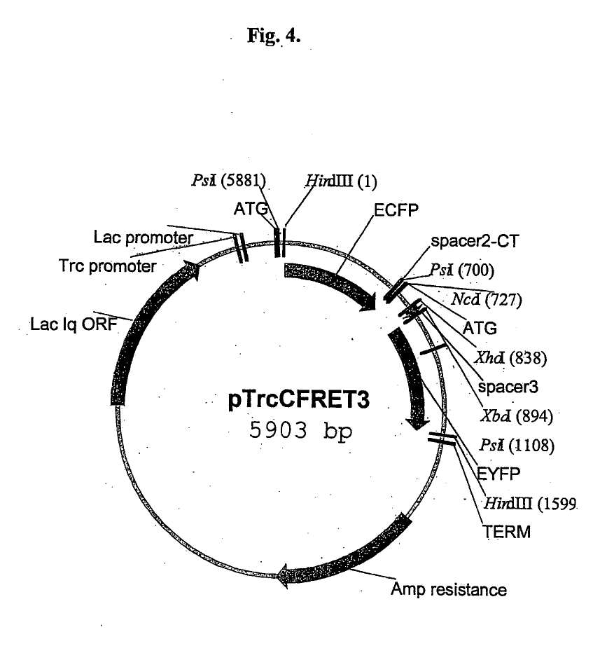

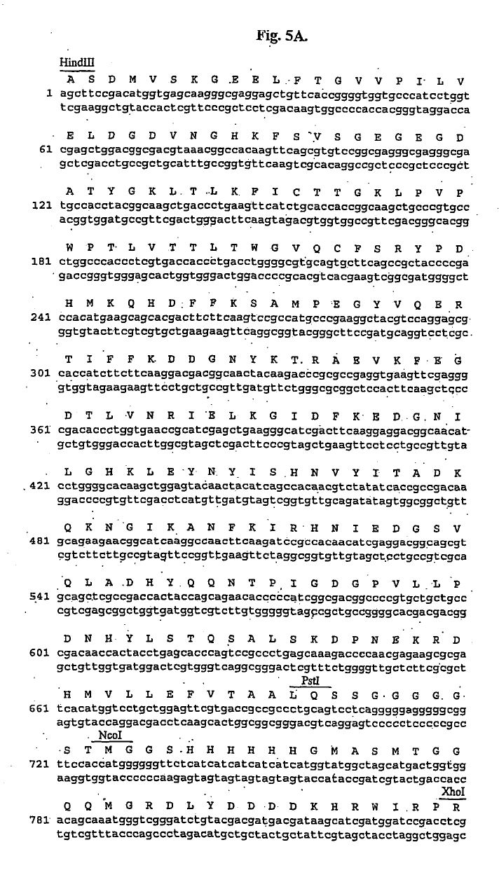

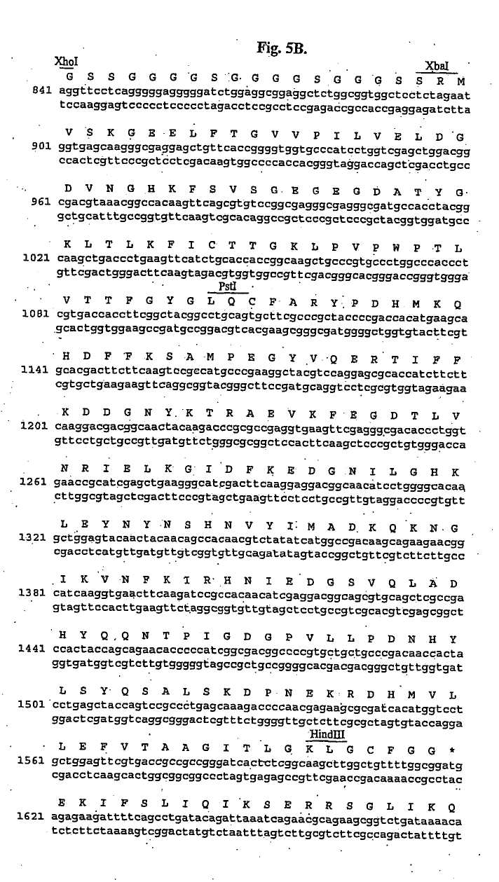

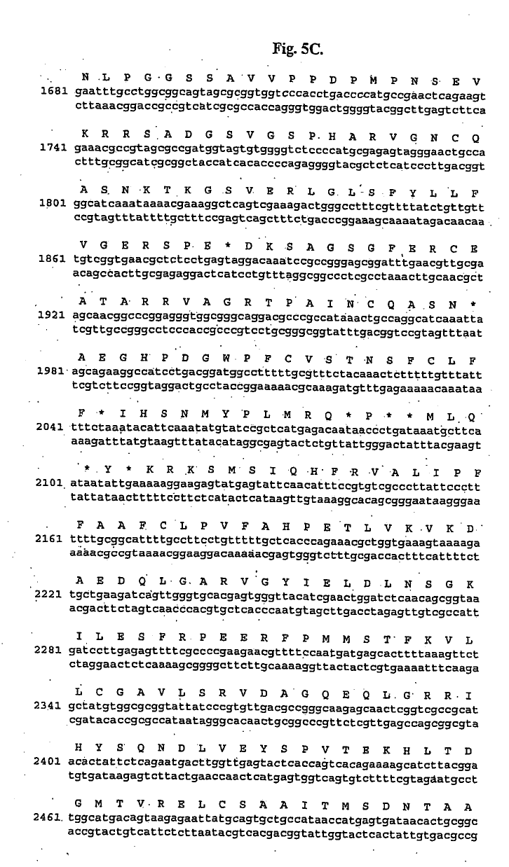

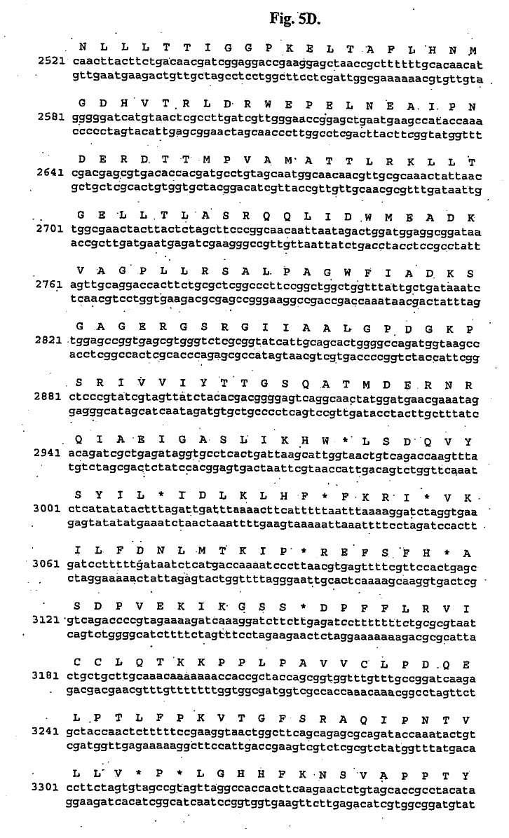

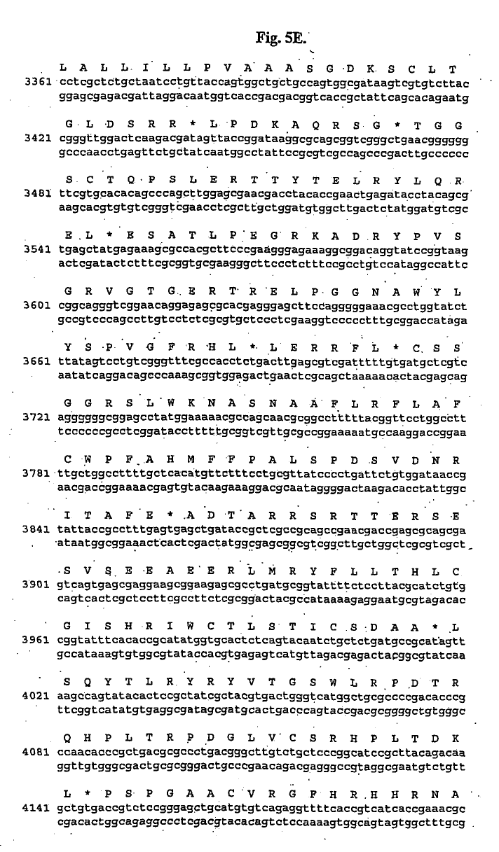

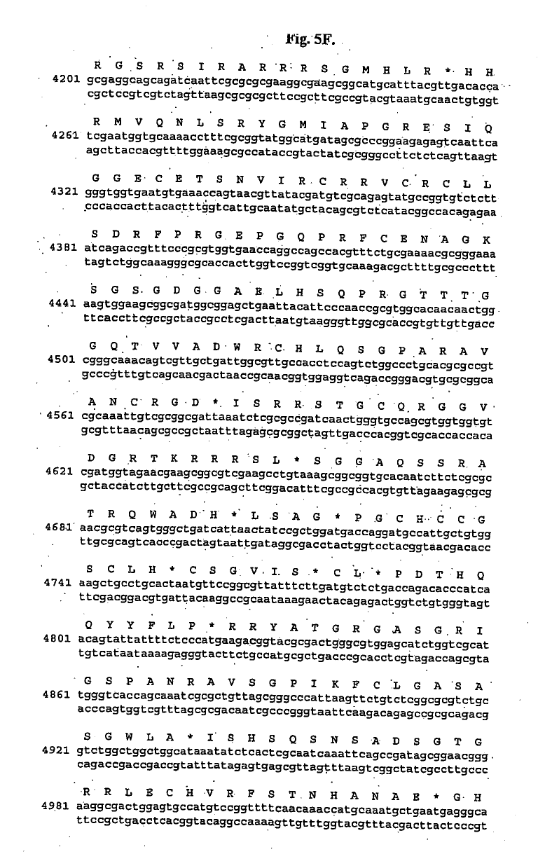

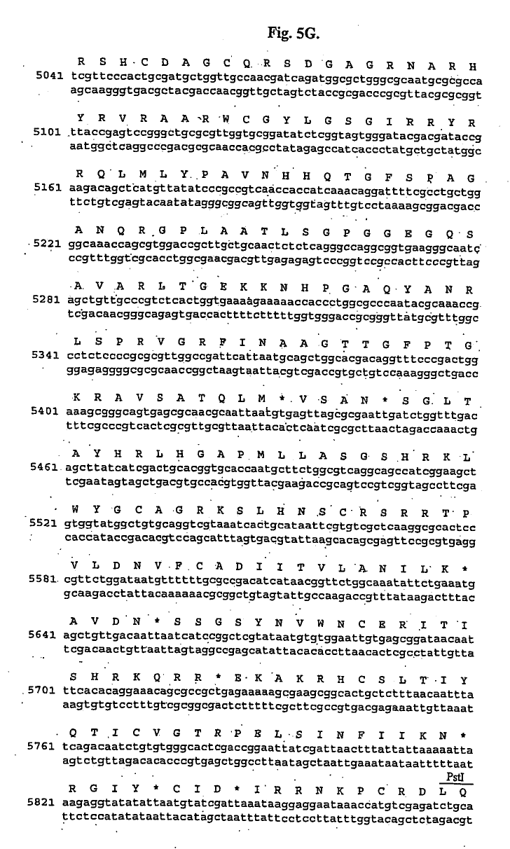

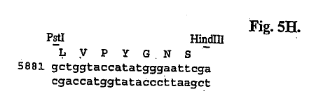

Plasmid pTrcCFRET3 was prepared. A schematic map of pTrcCFRET is set out in FIG. 4 and its sequence is set out in FIG. 5. Table 1 below sets out the features of pTrcCFRET3. The techniques and methodologies used in the preparation of pTrcCFERT3 were standard biochemical techniques. Examples of suitable general methodology textbooks include Sambrook et al., Molecular Cloning (1995), John Wiley & Sons, Inc.

TABLE 1
Feature table for pTrcCFRET3

| Nucleotide | Nucleotide | | |
|---|---|---|---|
| start | finish | Feature | Component |
| 11 | 13 | ATG | Initiator methionine for eCFP |
| 698 | 700 | CAG | Final residue of eCFP |
| 701 | 703 | TCC | Initial serine of spacer |
| 740 | 742 | CAT | First histidine of hexa-His tag |
| 758 | 760 | GGT | Glycine at start of epitope tag |
| 842 | 844 | GGT | Initial glycine of spacer |
| 899 | 901 | ATG | Initial methionine of eYFP |
| 1619 | 1621 | TGA | Termination codon for expression |

The whole of the pTrcCFERT3 construct contains a series of unique restriction sites for additional insertions, as shown on the plasmid map (FIG. 4) and is inserted into a HindIII cassette for ease of subcloning.

The construct is shown inserted into the mammalian expression vector pTrcHis (from which the multiple cloning site and internal His tag and cleavage sites have been removed). Transfer of this insert to any other expression system is facile for those skilled in the art.

1. A probe comprising:
(i) a target binding site moiety which is attached to a first fluorescent polypeptide;
(ii) a mimic moiety which is capable of binding to the target binding site moiety and which is attached to a second fluorescent polypeptide; and
(iii) a linker which connects the two fluorescent polypeptides and which allows the distance between said fluorescent polypeptides to vary, said fluorescent polypeptides being so as to display fluorescence resonance energy transfer (FRET) between them, wherein the linker comprises one or more of: (1) a sequence capable of being recognised and bound by an immobilized component; (2) a protease cleavage site; (3) a non-analyte binding site; (4) two or more copies of the sequence (SerGly₃); or (5) one or more copies of a rod domain from a structural protein.

2. A probe according to claim 1, wherein the target binding site moiety is a peptide.

3. A probe according to claim 1, wherein the mimic moiety is a peptide.

4. A probe according to claim 1, wherein the linker is a peptide.

5. A probe according to claim 1, wherein the entire probe is a single polypeptide.

6. A probe according to claim 1, wherein the sequence capable of being recognised and bound by an immobilized component is a His₆ tag, an antibody epitope, or a sequence recognised by a protein modification enzyme.

7. A probe according to claim 6, wherein the sequence recognised by a protein modification enzyme is a biotinylation site, a glycosylation site or a phosphorylation site.

8. A probe according to claim 1, wherein the protease cleavage site is an enterokinase or Factor X cleavage site

9. A probe according to claim 1, wherein the non-analyte binding site directs targetting of the probe to a sub-cellular localisation.

10. A probe according to claim 9, wherein the probe is targetted to the plasma membrane or nuclear envelope.

11. A probe according to claim 1, wherein the linker comprises from 2 to 4 copies of the sequence (SerGly₃).

12. A probe according to claim 1, wherein the linker comprises from 1 to 4 copies of a rod domain from a structural protein.

13. A probe according to claim 1, wherein the first fluorescent polypeptide is a green fluorescent protein (GFP).

14. A probe according to claim 1, wherein the second fluorescent polypeptide is a GFP.

15. A probe according to claim 13, wherein the first fluorescent polypeptide is cyan fluorescent protein (CFP) and the second fluorescent polypeptide is yellow fluorescent protein (YFP).

16. A probe according to claim 1, wherein the second fluorescent polypeptide is replaced with a non-fluorescent polypeptide.

17. A probe according to claim 1, wherein the mimic moiety comprises a peptide sequence capable of biotinylation.

18. A probe according to claim 17 which is biotinylated.

19. A polynucleotide which encodes a probe according to claim 5.

20. A polynucleotide according to claim 19 which is a DNA sequence.

21. A vector which incorporates a polynucleotide according to claim 19.

22. A vector according to claim 21, which is an expression vector.

23. A cell harbouring a probe according to claim 1, a polynucleotide according to claim 19 or a vector according to claim 21.

24. A fungus, plant or animal comprising a probe according to claim 1, a polynucleotide according to claim 19, a vector according to claim 21 or a cell according to claim 23.

25. A sensor comprising:
(i) a probe according to claim 1;
(ii) a light source which is capable of exciting the probe; and
(iii) a detector which is capable of measuring the amount of FRET from the probe.

26. A sensor according to claim 25, wherein there are two detectors, one of which is sensitive to the first fluorescent polypeptide of the probe and the other of which is sensitive to the second fluorescent polypeptide of the probe.

27. A sensor according to claim 25 which comprises more than one probe.

28. A method for detecting the presence or absence of a target substance in a test sample comprising:
(i) providing a probe according to claim 1, a cell according to claim 23 or a sensor according to 25, wherein the target binding site moiety of the probe, cell or sensor is capable of binding to the target substance;
(ii) determining the amount of FRET of the probe, cell or sensor;
(iii) contacting the probe, cell or sensor with the test sample; and
(iv) determining any change in FRET thereby to determine whether the test sample comprises the target substance.

29. (Canceled)

30. A method for identifying an inhibitor of binding between two substances, which two substances would bind to each other in the absence of an inhibitor, comprising:
(i) providing a probe according to claim 1, a cell according to claim 23 or a sensor according to claim 25, wherein the binding of the target binding site moiety of the probe, cell or sensor to the mimic moiety of the probe, cell or sensor mimics the binding of the two substances to each other;
(ii) determining the amount of FRET of the probe, cell or sensor;
(iii) contacting the probe, cell or sensor with a test substance; and
(iv) determining any change in FRET thereby to determine whether the test substance is an inhibitor of binding between the two substances.

31. (Canceled)
